(19) United States
(12) Patent Application Publication
(10) Pub. No.: US 2025/0027858 A1
(43) Pub. Date: Jan. 23, 2025

(54) CHARACTERIZATION OF MECHANICAL BIOMASS PARTICLE-PARTICLE AND PARTICLE-WALL INTERACTIONS

(71) Applicant: The Penn State Research Foundation, University PArk, PA (US)

(72) Inventors: James Charles Slosson, University Park, PA (US); Hojae Yi, University Park, PA (US)

(21) Appl. No.: 18/775,741

(22) Filed: Jul. 17, 2024

Related U.S. Application Data
(60) Provisional application No. 63/527,200, filed on Jul. 17, 2023.

(51) Int. Cl. G01N 3/10; G01N 3/24; G01N 33/483

(52) U.S. Cl. CPC G01N 3/10; G01N 3/24; G01N 33/4833; G01N 2203/0025; G01N 2203/0026; G01N 2203/0089; G01N 2203/0254; G01N 2203/0429

(57) ABSTRACT

Methods and systems facilitate measuring flow properties of a material, such as biomass materials, that include different material particles. Sample holding plates securely hold material particles and secure them to sample holders. A normal force actuator applies a normal force between material particles held by the sample holding plates and a sliding force actuator applies a sliding force. Normal and sliding force sensors monitor the transmitted forces, allowing a processor to determine adhesion and friction characteristics between particles held by the first and second sample holding plates.

### CROSS REFERENCE TO RELATED APPLICATION

This application claims the benefit of U.S. Provisional Patent Application No. 63/527,200, entitled “CHARACTERIZATION OF MECHANICAL BIOMASS PARTICLE-PARTICLE AND PARTICLE-WALL INTERACTIONS,” filed on Jul. 17, 2023, the disclosure of which is incorporated by reference in its entirety for all purposes.

### GOVERNMENT SUPPORT

This invention was made with government support under Grant No. DE-EE0008936 awarded by the Department of Energy and under Hatch Act Project No. PEN04671 awarded by the United States Department of Agriculture/NIFA. The Government has certain rights in the invention.

### TECHNOLOGY FIELD

The present disclosure relates to characterizing mechanical properties of heterogeneous feedstock, such as corn stover and other crop residues, such as forest residues or mulch or non-biological heterogeneous feedstocks for industrial applications.

### BACKGROUND

Crop residue, such as corn stover, is a key candidate as a biomass source for its abundance and current use in integrated biorefineries. Corn stover refers to the remaining heterogeneous corn plant fractions (i.e., stalk, husk, leaf, and cob) after the lower stalk is cut away and the grain has been harvested. While many want to bring the advantages of new biofuel technologies to industrial production, there is a significant financial risk due to frequent misrepresentation of physical and chemical variabilities in effective lignocellulosic biomass handling and feeding. As of 2016, total biofuel production only reached 7% of the expected 58 billion gallons per year design capacity. The challenges in the handling of biomass are one of the crucial impediments to adopting biomass as feedstocks for biofuel production. Understanding interparticle mechanics associated with each corn stover fraction will provide valuable information for understanding the overall poor flow behavior of milled corn stover. More specifically, such quantitative information will help identify certain fractions that may be significantly responsible for the poor flowability behavior of comingled corn stover fractions. Similar issues arise in other heterogeneous feedstocks, such as pine forest residues (which can include heterogeneous fractions of wood chips, needles, bark, and twigs).

The challenges in the handling of biomass are one of the crucial impediments to adopting biomass as feedstocks for biofuel production. Difficulties in handling these feedstocks arise from the variation in particle interactions within and among the fractions of different materials within the feedstock, which affects the free flow of material. Different fractions of material can result in different or erratic flow properties.

Flowability challenges are mainly associated with biomass density, particle size distribution, moisture content, angle of repose, shear stress, bridging tendency, cohesive strength, and friction of equipment surfaces. The variations in biomass factors such as shape, size, moisture content, and low bulk density often make it challenging to handle and transport in its original form. However, the majority of the metrics used for the mentioned flow challenges are focused on bulk behavior and do not address the inter-particle mechanics leading to bulk observation.

Understanding interparticle mechanics associated with each corn stover fraction will provide valuable information for understanding the overall poor flow behavior of milled corn stover. More specifically, such quantitative information will help identify certain fractions that may be significantly responsible for the poor flowability behavior of comingled corn stover fractions.

There is a need to better understand how different fractions of material can affect the flow characteristics of a heterogeneous feedstock.

### SUMMARY

Embodiments of the present invention address and overcome one or more of the above shortcomings and drawbacks by providing systems and methods to apply a normal force and a sliding force between two material particles and observing and characterizing the interparticle forces.

In one embodiment, a measurement system for measuring flow properties of a material having a plurality of material particles includes a plurality of sample holding plates, each configured to securely hold a material particle. A first and a second sample holder are each configured to securely hold a first and second one of the plurality of sample holding plates, the first sample holder being fixed during a measurement. A normal force actuator is configured to apply a normal force between material particles held by the first and second sample holding plates. A sliding force actuator is configured to apply a sliding force between the first and second sample holding plates. A normal force sensor is configured to determine the normal force. A sliding force sensor is configured to determine an amount of sliding force transmitted from the sliding force actuator through the material particles held by the first and second sample holding plates. A processor is configured to control the normal force and sliding force actuators, receive measurements from the normal force and sliding force sensors, and determine adhesion and friction characteristics between material particles held by the first and second sample holding plates.

In one embodiment, a method for measuring flow properties of a material having a plurality of material particles includes steps of preparing a plurality of sample holding plates, each securely holding a material particle, securing a first and second one of the plurality of sample holding plates to a first and second sample holder, and operating a processor. The processor is operated to apply a normal force between material particles held by the first and second sample holding plates using a normal force actuator, apply a sliding force between the first and second sample holding plates using a sliding force actuator, receive measurements from the normal force and sliding force sensors, where the sliding force sensor detecting an amount of sliding force transmitted from the sliding force actuator through the material particles held by the first and second sample holding plates, and determine adhesion and friction characteristics between material particles held by the first and second sample holding plates.

In one embodiment, a measurement system for measuring flow properties of a material having a plurality of material particles includes a first and a second sample holder, each configured to securely hold a material particle during a measurement. A normal force actuator is configured to apply a normal force between the material particles. A sliding force actuator is configured to apply a sliding force between the material particles. A processor is configured to control the normal force and sliding force actuators, receive measurements from one or more force sensors, and determine adhesion and friction characteristics between the material particles.

According to aspects of some embodiments, each of the plurality of sample holding plates are configured to hold material particles using an adhesive. In some embodiments, the adhesive is a cyanoacrylate glue. In some embodiments, each of the plurality of sample holding plates have a mating face that is keyed such that it slides into each sample holder. In some embodiments, each of the plurality of sample holding plates have a mating face that is keyed such that it is configured to mount to each sample holder in a plurality of predetermined orientations to facilitate testing the same particle in multiple selectable orientations. In some embodiments, the processor is further configured to store the adhesion and friction characteristics for multiple combinations of the plurality of sample holding plates and to create a statistical model of interparticle forces, wherein the model divides material particles into predetermined particle types and identifies force characteristics for each particle type. In some embodiments, the material is corn stover and the plurality of material particles comprise pieces of cob, husk, leaf, and stalk. In some embodiments, the material is forest residue and the plurality of material particles comprise wood chips, bark, needles, and twigs.

### BRIEF DESCRIPTION OF THE DRAWINGS

FIG. 1 is a logical relationship diagram explaining the relationship between different scales of flowable materials;

FIG. 2A is a side view of the particle interaction section of an exemplary inter-particle mechanics tester (IPMT) for use with some embodiments;

FIG. 2B is a side view of an exemplary IPMT for use with some embodiments;

FIG. 3A is a top view of various samples of corn stover materials secured to contact plates;

FIG. 3B is multiple side and head-on views of various mating face designs to secure contact plates to sample holder structures;

FIG. 4 is a conceptual diagram of the various interparticle forces in a flowable material;

FIGS. 5 and 6 are exemplary test data from using an exemplary IPMT on different particles;

FIG. 7 is a system diagram of the electrical components of an exemplary IPMT for use with some embodiments; and

FIG. 8 is a flow chart for using an exemplary IPMT in accordance with some embodiments.

### DETAILED DESCRIPTION

Embodiments disclosed herein aim to gain quantitative knowledge of the interparticle mechanics of fractionated corn stover through the development of an interparticle mechanics tester capable of accommodating flowable materials having variable particles (e.g., biomass particles, such as corn stover or forest matter) and a test protocol to determine friction and adhesion properties. For example, with the novel data on interparticle mechanical properties of biomass materials, such as corn stover particles from different anatomical origins, biofuel and material handling engineers could benefit from examining the significance of the difference in friction coefficients between corn stover particles from different anatomical fractions. The knowledge gained by testing will contribute to reducing operational challenges by removing fractions with high friction and traction adhesion contributions.

The flow characteristics of the material are an aggregate of the particle interactions that make up the bulk of the material. Thus, by providing reliable testing methods and apparatus to determine which subfractions of material contribute to restrictions in the flow of heterogeneous bulk material, one can determine whether different components of the feedstock may need to be removed or the fraction reduced to achieve the desired flow characteristics for a given feedstock for a given processing and material handling system and method. Alternatively, by understanding the effect different subfractions have on the flow of material, handling systems can be selected or designed for a given material aggregate composition being handled.

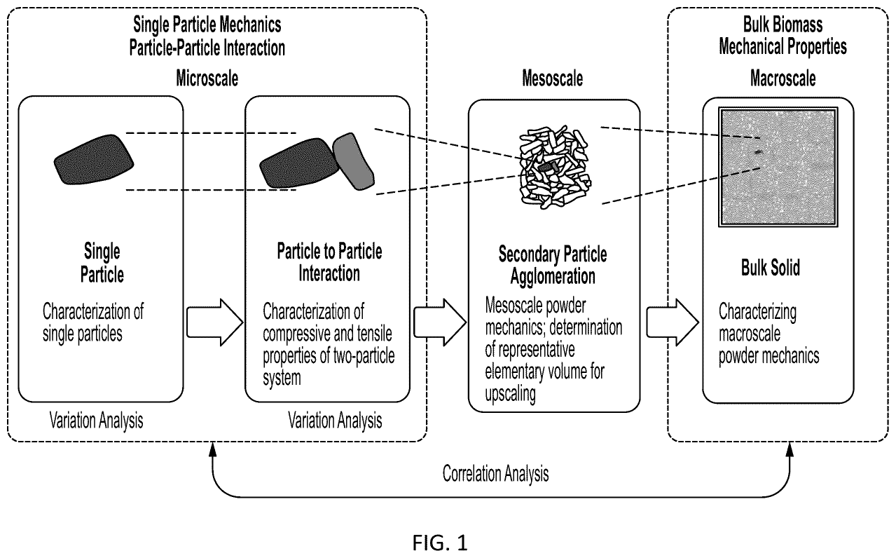

Exemplary goals for a particle-interaction characterization method and apparatus include: developing an experimental method to characterize the friction and adhesion of material particles; determining friction and adhesion properties, and their variabilities of feedstock (e.g., southern pine residue and corn stover) particles relative to tissue type; analyzing correlation between bulk flow properties and the friction coefficient and adhesion force; determining friction coefficient and adhesion forces that are crucial parameters of the discrete element modeling. FIG. 1 shows the high-level explanation of the interplay between the individual particles within a flowing material and the bulk flow properties of a material. The focus of some embodiments is on improving characterization of single particle and particle-particle interactions. These methods can be used to determine which particle interactions are most directly impacting flow characteristics of bulk material, allowing handlers to determine which fractions of materials should be removed if bulk characteristics are outside of parameters for a given process.

Exemplary methods and apparatus described herein can characterize material with a particle size between 0.1 and 10.0 mm. In some embodiments, particles can be 1-50 mm, depending on the material being characterized.

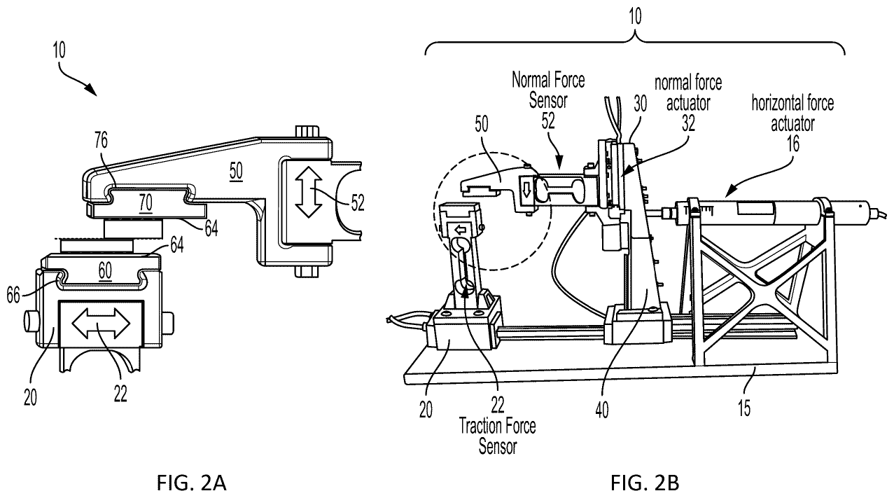

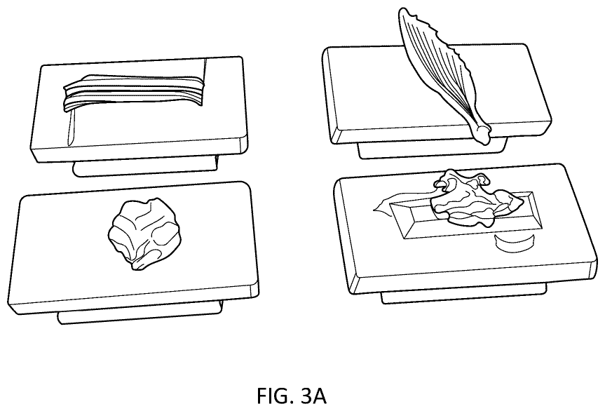

To characterize particle interactions, an inter-particle mechanics tester (IPMT) was developed (FIGS. 2A, 2B) to measure the traction force between a stationary and moving particle under varying magnitudes of normal load. This arrangement allows for one test run to produce multiple normal force and lateral force measurements and alleviates the requirement to conduct separate tests with different normal forces in a conventional friction test. The development of this IPMT allows simulation of many orientations of two interacting particles. This design allows collecting friction coefficients over multiple untested regions without resampling surfaces or replacing test particles between each repetition. With this design, it is also possible to prepare and condition multiple test samples, e.g., conduct friction experiments in an environment-controlled chamber at a different environmental relative humidity.

An exemplary IPMT (FIGS. 2A, 2B) can perform friction tests on fully supported particles, unsupported (over-hanging), or combinations of both. Sample holders and component position/capacity are easily adjustable to handle any particles of any size and shape. The size and characteristics of the hardware used in the IPMT can be adjusted to meet any other suitable requirements depending on the particles being tested. Exemplary test condition with an exemplary IPMT include: minimum incremental push/pull motion of 0.05 μm; maximum push/pull travel ranges up to 25 mm to accommodate a typical biomass particle size; push/pull velocity is set to 1.0-1.5 mm/s; maximum displacement of the normal force actuator is 14 mm to accommodate a typical biomass particle size; minimum normal displacement of the particle is as small as 6 nm; three-dimensional control of the initial particle contact location is possible; maximum allowed normal force is approximately 3 N; accuracy of loadcell is +0.02% of FS; and maximum sample rate is 1 kHz. In these examples, a typical biomass particle includes material particles from crop residue that is commonly handled for a biofuel plant. Other material particle sizes can be assumed for other applications.

The exemplary IPMT 10 in FIGS. 2A and 2B includes two stationary components (a first/lower sample holder 20 and a force applicator housing 15) and a gantry 30 that can slide horizontally relative to the stationary components to apply traction or a pushing force between a particle mounted to sample holder 20 and one mounted to gantry 30. Gantry 30 can be moved horizontally under test (towards or away from housing 15) and includes a horizontally sliding support 40 and vertically sliding second/upper sample holder 50. Horizontally sliding support 40 slides horizontally toward and away from housing 15, while vertically sliding second sample holder 50 is supported by horizontally sliding support 40. Applying a vertical (downward, compressive relative to the sample holders) force to sample holder 50 applies a normal force between two sample plates, one rigidly coupled to second sample holder 50 and another rigidly coupled to first sample holder 20. First sample holder 20 includes a traction force sensor (load cell) that detects the horizontal force applied to the stationary particle(s) under test, such as by measuring a torsion force on a support portion of sample holder 20 or a sheer force between the rigid support and a plate that holds the sample.

Force applicator housing 15 is a rigid housing or base that supports a linear actuator (horizontal sliding force actuator 16) that applies a horizontal sliding force (traction/pushing force) to the horizontal sliding gantry 30 (moving sliding support 40). Support 40 of gantry 30 includes another linear actuator (normal force actuator 32) oriented vertically to apply a downward force to create a normal force between a first contact plate 60 that fixedly holds one particle or a group of particles to first sample holder 20 and a second sample contact plate 70 that fixedly holds one particle or a group of particles to second sample holder 50. Second sample holder 50 can include a force sensor/load cell (normal force sensor 52) that detects a sheer or torsion force between the distal end of second sample holder 50 (which holds sample contact plate 70) and the proximal end (mounted to normal force actuator 32) to determine the normal force applied between the sample contact plates 70 and 60. Horizontal sliding force actuator 16 applies a horizontal traction or pulling force to create a sheer force between the sample plates. Traction force sensor 22 measures the sheer force between the samples to determine friction and adhesion characteristics for the particles at the contact plane between them. Both force sensors can be any type of force sensor, such as a piezoelectric sensors that measures linear or torsional displacement in within the respective sample holders 20 and 50.

In some embodiments, a position encoder or accelerometer can be used with support 40 or sample holder 50 to determine the exact motion profile between the plates for additional data. This can be accomplished, for example, by using an actuator with a real-time position encoder that provides motion feedback. This allows observation of how much a particle moves for a given force input or load. In some embodiments a load cell or current and voltage sensor is used in the drive circuit of the sliding force actuator to determine the input traction/pushing force to the gantry. Real-time data is collected from load cells to determine a normal force and a friction force between the plates, and the total traction applies. The ratios between the normal force (measured by the normal force sensor) and the friction force (measured by the traction force sensor) can then be used to determine the inter-particle coefficient of friction. The adhesion force between the particles on the two plates is the force that is not affected by the normal force. By plotting the traction force observed by the sensor in the first sample holder 20 vs the normal force, the drag force between the plates can be broken down into the force from the coefficient of friction (proportional to the normal force) and the adhesion force between the materials.

Sample contact plates 60 and 70 each have a contact face (64 and 74, respectively) that each hold a sample and a mating face (66 and 76, respectively) that allows the contact plate to be secured to the respective sample holder structure (20 and 50, respectively). In some embodiments, mating faces 66 and 76 are keyed to allow them to be easily mounted the sample holder without tools or fasteners. During normal use, dozens of samples will be used to fully investigate the inter-particle adhesion and friction forces to establish a statistically relevant model. Accordingly, the mating faces should be designed to allow a fast swapping of sample contact plates. In this example, sample contact plates 60 and 70 are disposable 3D printed or injection-molded plastic pieces with a dovetail design to the mating faces to allow them to be quickly manufactured at low cost so that particles can be glued (e.g., using cyanoacrylate or epoxy) to the contact face and the mating face allows the plate to be easily slid into sample holders 20 and 50. The dovetail design holds it securely with a friction fit. In some embodiments, a spring or latch adds additional security to hold each contact plate.

### Experimental Analysis

An exemplary embodiment was tested with dozens of particles of corn stover to demonstrate how an IPMT can measure and statistically characterize inter particle forces. A square bale of field-dried corn stover from Antares Corn Stover (Hardin, IO) was processed by Forest Concepts, LLC (Auburn, WA). The bale was hand separated and placed into labeled bins designated for each anatomical fraction. Individual fractions were then comminuted to Crumbles® using Forest Concepts' Crumbler® Rotary Shear technology and conveyed into a final screening process. The screening was performed using a 3-deck orbital screen to filter out fractions with geometric mean diameter outside the 4 mm target.

Lab characterization for anatomical fractions of corn stover was provided by Forest Concepts, LLC analytics laboratory in Auburn, WA. Final products were analyzed for size, moisture content, and loose and bulk density (Table 1). Individual corn stover fractions were packaged and shipped to Pennsylvania State University's Food and Biomaterials Lab for inter-particle mechanics testing.

TABLE 1
Lab characterization for anatomical fractions of corn stover. Provided
by Forest Concepts, LLC analytics laboratory in Auburn, WA.

| | Moisture | Geometric Mean | | Bulk Density | |
|---|---|---|---|---|---|
| Anatomical | Content | Diameter (mm) | | (odkg/m\hat{ }3) | |
| Fraction | % MCwb | Xgm | Sgm | Loose | Tapped |
| Cob | 9.7 | 6.6 | 1.6 | 179 | 217 |
| Husk | 9.7 | 4.6 | 1.6 | 48 | 66 |
| Stalk | 9.7 | 4.5 | 1.8 | 76 | 98 |

An experimental procedure was developed to perform friction tests with minimal experimental error, as detailed below. A sample size of 12 was selected for each possible combination of anatomical fractions to obtain a minimum of three data points and to ensure the experiment could be completed as planned. Once all the data had been collected and analyzed, an analysis of variance (ANOVA) (alpha=0.05) was used to determine the source of variance. The Wilcoxon signed-rank (signed-rank) test was selected to test the null hypothesis (i.e., friction coefficients between corn stover particles from different anatomical fractions are not statistically different) for a population of unknown statistical distribution. The signed-rank test is a useful alternative to a paired student t-test when the normality of the observed data set is not ensured.

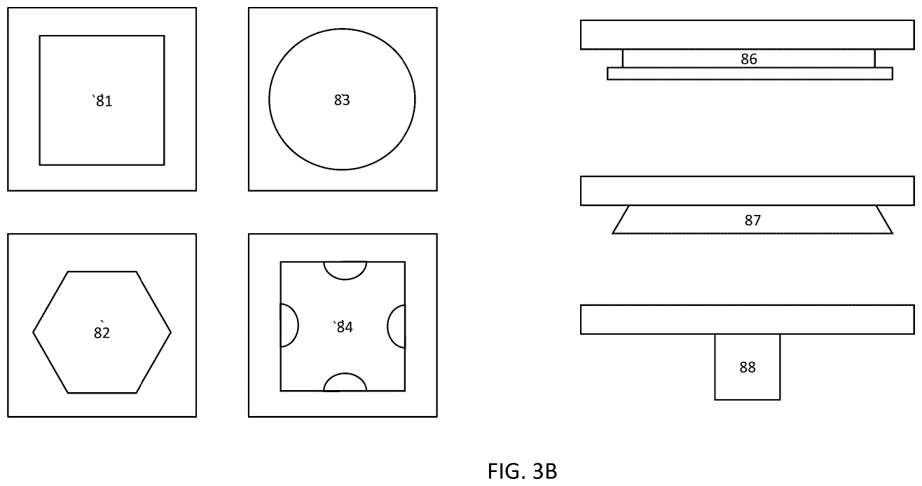

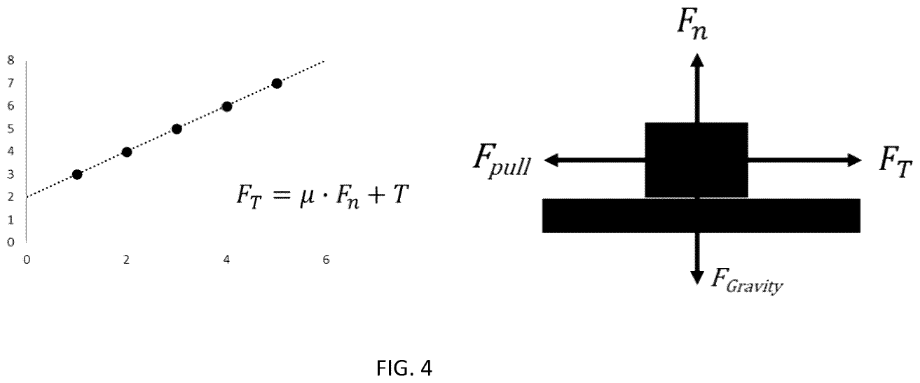

Approximately 50 g samples were collected from each anatomical fraction of corn stover. Each sample was held in a controlled environment for 72 hours to allow for particles to reach equilibrium moisture content (˜10% MCwb). Once particle moisture was equalized, particles were randomly selected and adhered to removable particle mounts (FIG. 3) using an adhesive, such as Permatex Ultra Bond™ Super Glue (cyanoacrylate glue). Suitable adhesives are bonding agents that affix material particles to the mount in a permanent or temporary manner such that the material particle is held securely in one place during each test without moving relative to the mounting substrate. Mounting methods (fully vs. singly supported) and grain orientations were performed randomly with every particle. Conservative volumes of adhesives were applied to reduce absorption into particles as well as to ensure proper curing. A curing period of 24 hours (+2 hours) was established for experimental consistency. Once the adhesive had cured for 24 hours, particle pairs were randomly selected for testing. Table 2 contains the experimental plan for each possible particle pair and the required number of repetitions to be completed to test the null hypothesis.

FIG. 3A shows exemplary test plates to be used as a sample contact plate 60 or 70 in the IPMT of FIG. 2. FIG. 3A is an image of corn stover particles adhered to plastic particle mounts for conditioning and testing. The upper and lower half of the image contains 4 mm husk and cob fractions, respectively.

FIG. 3B shows some exemplary profiles for the mating faces of exemplary contact plates. These exemplary mating faces can be used as the contact faces of contact plates 60 and 70. Faces 81, 82, 83, and 84 are head-on views looking into the mating face. Mating face 81 is square-shaped, allowing four different orientations of the plate relative to the sample support it is mating to. Mating face 82 is hexagonal, allowing six different orientations. Other shapes having any number of sides can be easily used, as well. 83 is circular shaped, allowing an arbitrary number of orientations to be used. Mating face 84 includes some exaggerated detents that can be molded into the face that allow the mating face to positively engage with corresponding nubs in the sample support, such that the contact plate is more securely mounted. The corresponding nubs in the support structures can be spring-loaded or flexible to allow the mating face to be easily inserted and withdrawn with moderate force.

Mating faces 86 through 88 are side views, such as shown in FIG. 2A. Face 86 is t-shaped, while face 87 is dovetail shaped, such as is shown in FIG. 2A. These are designed to be slid into the support structure from the side. Mating face 88 is an elongated member designed to be inserted in the direction of the normal force into the support structure. This elongated member can have the profiles shown in faces 81 through 84 or may be threaded, depending on the application. Each of these mating faces allow quick and secure attachment between the contact plate and the sample holding structure. Not shown in these examples is the particles under test, which would be glued or clamped to the contact face of the contact plate.

TABLE 2
Experimental plan for the required number of corn
fraction test pairs and associated repetitions.

| Pair ID# | Base Particle | Interacting Particle | Req. Test Repetitions |
|---|---|---|---|
| 1 | Stalk | Stalk | 12 |
| 2 | | Cob | 12 |
| 3 | | Husk | 12 |
| 4 | Cob | Cob | 12 |
| 5 | | Husk | 12 |
| 6 | Husk | Husk | 12 |

Each experiment was performed at a sliding speed of 0.5 mm/min with a randomized initial normal loading force between 30 and 200 g. Prior to each test, test operators should determine the required positioning of the normal force applicator (See FIG. 2) to produce the target initial normal loading. This procedure was performed in less than 0.1 mm increments to prevent overloading of the normal force sensor past the selected design capacity. Once the target initial normal force was applied, the traction force applicator and data collection were initiated. Data collection is terminated once particles are no longer in contact or upon sensor capacity concerns.

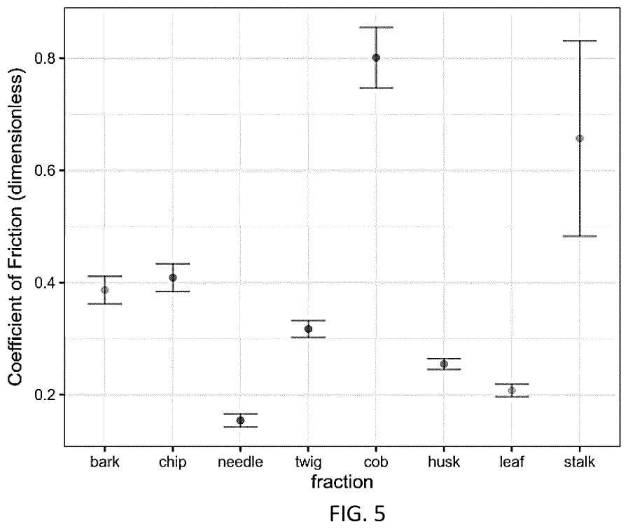

Normal and traction force data were collected at 80 Hz, using National Instruments™ Lab VIEW and two 24-bit Di-1000U signal digitizers (Loadstar Sensors-Freemont, CA). The middle two quartiles were selected and extracted as steady-state data for further analysis for each data collection experiment performed. FIG. 4 is a theoretical plot of traction force versus normal force (left) and a force diagram of two particles (rectangles) under applied normal and pulling force (right). The coefficient of friction (u) can be determined using the average ratio of traction force to the applied normal force during steady-state conditions. T is the adhesion force, which resists the sliding force, independent of the normal force.

### Results

Corn stover interactions involving cob fractions were found to contain the largest peaks and variance in friction coefficients (Table 3). Cob particles with woody-ring sub-fractions on the contacting surface were observed to be significantly harder and ‘claw-like’ in nature. Whereas the pith region of the cob was very similar to the exposed pith in stalk particles. Particles of stalk and husk both showed moderate friction coefficients.

TABLE 3
Average and standard deviations for each combination
of interacting corn stover fractions.

| Paired Fractions | Average Friction Coefficient | Standard Deviation |
|---|---|---|
| stalk-stalk | 0.28 | 0.09 |
| cob-cob | 0.86 | 0.26 |
| husk-husk | 0.23 | 0.06 |
| stalk-cob | 0.53 | 0.25 |
| cob-husk | 0.32 | 0.16 |
| stalk-husk | 0.21 | 0.07 |

TABLE 4
Wilcoxon Signed Rank Test Critical Values Table

| Particle Pair | Comparison Pair | Sum < Cr? | Result |
|---|---|---|---|
| stalk-stalk | cob-cob | TRUE | reject null |
| | husk-husk | FALSE | fail to reject |
| | stalk-cob | TRUE | reject null |
| | cob-husk | FALSE | fail to reject |
| | stalk-husk | FALSE | fail to reject |
| cob-cob | husk-husk | TRUE | reject null |
| | stalk-cob | TRUE | reject null |
| | cob-husk | TRUE | reject null |
| | stalk-husk | TRUE | reject null |
| husk-husk | stalk-cob | TRUE | reject null |
| | cob-husk | FALSE | fail to reject |
| | stalk-husk | FALSE | fail to reject |
| stalk-cob | cob-husk | FALSE | fail to reject |
| | stalk-husk | TRUE | reject null |
| cob-husk | stalk-husk | TRUE | reject null |

An ANOVA test (α=0.05) showed the key source of variance in friction coefficients was found to be between different combinations of fractions. Leading to further investigation of key sources of variances. Without an understanding of the population distribution, a Wilcoxon signed-rank test was conducted to determine the significance across the variance with each corn stover fraction combination. As shown in Table 4, the results suggest that cob fractions had a significantly different friction coefficient.

From the observation that particles of corn cob show a larger standard deviation in the determined coefficient of friction, it can be hypothesized that particles of corn cob will have a high contribution to an erratic feedstock flow behavior. It can also be hypothesized that the cob will generate high frictional forces considering the high cohesive forces and moderate friction coefficients observed in a corn cob. The experimental data suggest that high-stress feedstock handling applications may be more stable with the removal of corn cob.

It is planned to repeat tests with variations in moisture content. High adhesive forces in cob particles containing ‘woody-ring’ subfractions may be reduced as moisture levels increase towards the fiber saturation point, but further studies are required to understand any adverse effects on friction coefficients.

The anatomical fractions discussed in this paper were categorized using macro-observations for the purpose of potential mechanical separation. Under further review, it appears each corn stover fraction contained an associated set of sub-fractions that could be found in at least one other fraction. Sub-fractions could be generally characterized as woody, pithy, or leafy. Additional steps in comminuting particles to expose ‘sub-fractions’ may allow fluid separation techniques to create more uniform and, therefore, desirable products. It is recommended to repeat this experiment using two additional comminution methods (i.e., knife mill and hammer mill) practiced in industry to study varying effects of size reduction equipment on friction coefficients.

Embodiments of an IPMT can be used to test particle interactions within biomass feedstock or other flowing materials, such as recycling feedstock (e.g., plastic or metal scrap). This can be particularly useful for addressing biomass feedstock handling challenges in producing advanced bioenergy from terrestrial biomass. The IPMT can expand the modeling capability (Discrete Element Modeling efforts in handling of particulate materials beyond biomass. The IPMT can produce quantitative data that can be used as a quality assurance metric of biomass feedstocks and increase fundamental knowledge on the origin of flowability issue that can guide a decision making to improve the reliability of biomass handling.

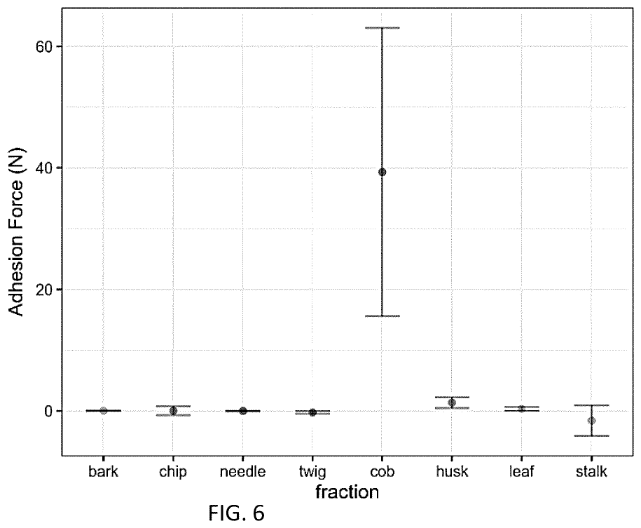

FIG. 5 shows exemplary friction coefficients found experimentally with wood residue and corn stover residue. Cob and stalk provide the highest coefficients of friction within corn stover. Needles provide the lowest among pine residue particles. FIG. 6 shows exemplary corresponding results for the adhesion forces.

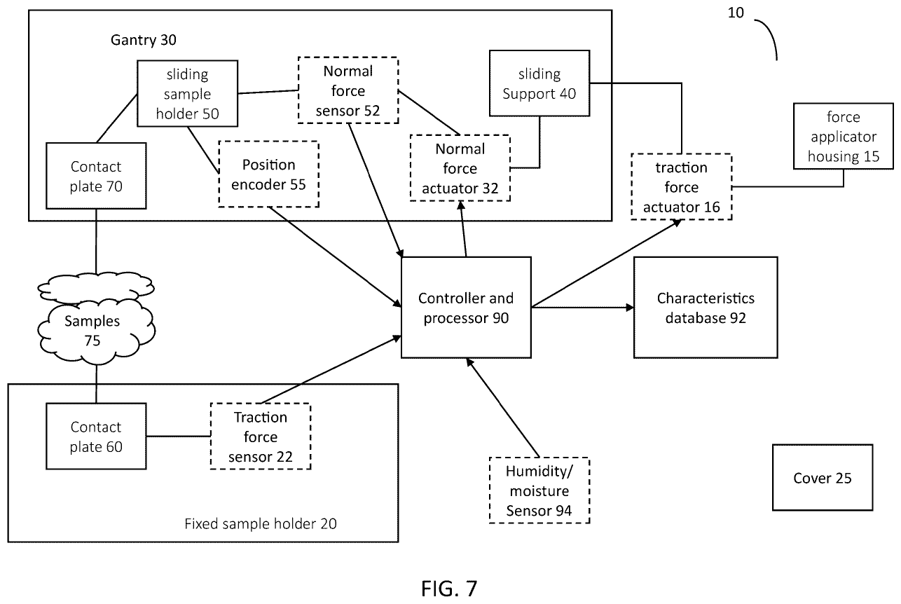

FIG. 7 is an exemplary system diagram for an IPMT showing the control outputs and sensor inputs for a processor/controller providing test stimuli and recording data for the interaction of two sample plates in accordance with the exemplary IPMT of FIGS. 2A and 2B. A position encoder is an optional component. The characteristics database can be used to collect sample results from a number of samples to create a statistical profile, such as shown in the foregoing results.

FIG. 7 shows the interaction of the mechanical and electrical components of an IPMT. Electrical and electromechanical components that interact with controller 90 are shown with dotted outlines. Controller 90 includes a processor (including software instructions and memory) and the appropriate drive and receiver circuits allowing this interaction. Controller 90 controls the operation of sliding force actuator 16 to apply a pushing or traction force between force applicator housing 15 and sliding support 40. This allows sliding support 40 to move relative to force applicator housing 15 under processor/software control. A normal force is applied between sliding sample holder 50 and sliding support 40 via processor/software control, using normal force actuator 32. The applied normal force is monitored by normal force sensor 52, which sends the data stream into controller 90. Optionally, sliding sample holder 50 includes position encoder 55. This encoder 55 can optionally be an accelerometer. This allows controller 90 to model forces by observing the acceleration and movement of sliding sample holder 50. Sliding sample holder 50 holds contact plate 70. One of samples 75 is glued or clamped to contact plate 70, allowing it to be pushed or pulled relative to a corresponding sample that is glued or clamped to contact plate 60.

Traction force sensor 22 within fixed sample holder 20 observes the deflection of contact plate 60 relative to the main body of IPMT 10. This deflection is based on the interactions between samples 75. A data stream from traction force sensor 22 is sent to controller 90. Controller 90 then calculates inter-particle characteristics from the information observed from the interplay of the force actuators and sensors into characteristic database 92 (including calculating friction coefficients and adhesion forces). This allows simply swapping samples 75 or reorienting their contact plates to generate a large number of samples for the friction and adhesion coefficients within characteristic database 92. By swapping out or reorienting samples 75 and rerunning the process under control of controller 90, dozens of data points can be quickly generated. This allows a statistically relevant model of force coefficients to be generated under processor control with minimal user interaction beyond swapping and reorienting contact plates.

Optionally, IPMT 10 can include a cover 25 with a door that allows the test environment to be controlled. This allows temperature and humidity to be set for each test. In practice, samples ready for test can be fully hydrated and weighed and dehydrated and weighed separately from the IPMT, which allows the moisture content and moisture-holding capabilities of each sample to be determined. For a given test, samples can be hydrated to any level within this range before testing to determine the impact of moisture content on the friction and adhesion properties. The samples can be stored at a given temperature and humidity within the housing of the IPMT or in a separate storage location until testing. A heater and a humidifier or dehumidifier can be used with cover 25 to ensure that the testing environment is close to the storage conditions. Sensors or input can tell processor 90 the environmental details so that temperature, weight, and moisture content can be included in the data stored in characteristics database 92.

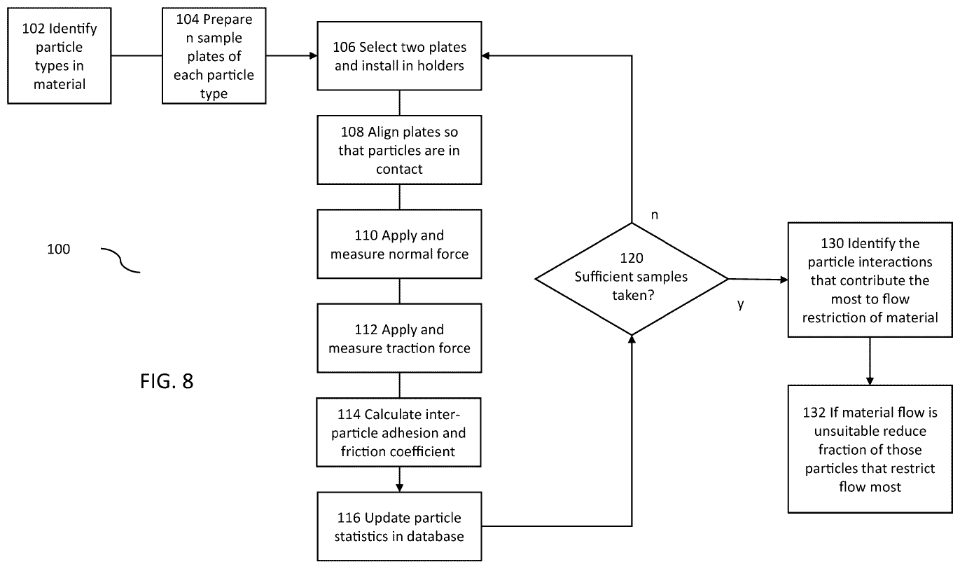

FIG. 8 is an exemplary flowchart for using an IPMT to evaluate particle interactions in any heterogeneous flowing material. Once sample plates have been created in accordance with the examples above, sample combinations can be used to collect statistical data to identify the important trends. Once the particle interactions have been characterized, this information can be used to troubleshoot material flow such as within a bioreactor using corn stover feedstock. Similarly, this process can be used to quickly characterize other heterogeneous materials, such as inorganic recycling or organic feedstock.

Exemplary characterization process 100 begins at step 102, where various particle types are identified in the material. If the biomass is corn stover, this includes pieces of husk, etc. If the biomass is forest matter, particles can include wood chips, bark, sticks, leaf matter, etc. This determination will vary depending on the application. At step 104, a sample size for particle interactions is determined and a sufficient number of particle samples are prepared. Testing has revealed that for many biomass types a test sample size “n” around 15 for each combination of particle types should be sufficient to give statistically relevant data. (For a biomass having k particle types, this would include n×k! total tests, or 150 tests for four particle types, which includes ten combinations) At least n+1 contact plates of each particle type are prepared with samples of each type of particle. Preparation can include gluing or clamping the particle to each contact plate, drying or hydrating each particle to an appropriate moisture content, and storing the mounted sample to maintain moisture and temperature. Each prepared sample can include an identification tag that can be correlated with the particle type, weight, moisture content, origin, and any other information. This identifier can be used to correlate characteristics in the characteristics database. In some embodiments, different mounting techniques can be used to fully or partially mount the particle. Fully mounted particles can be stabilized so that the friction surface is rigidly coupled to the mounting plate to minimize stretching or shearing effect within the particle. Partially mounted particles affix the face opposite of the friction face being tested without additional support or stabilization. This allow the particle to move or stretch internally under the shearing stress of the friction.

At step 106, two contact plates that have been prepared with two particle samples are installed into the sample holders, such as aligning the keyed mounting face of each contact plate into the corresponding receptacle in the sample holder. An identification of each particle sample can be input through a software user interface to controller 90 or barcode/QR scanner, to perform the interparticle measurement and associate that data with particle characteristics in the database.

At step 108, the IPMT can be set up for the test by sliding the support 40 to an appropriate location manually or under processor control. At step 110, when the particles are in position, processor 90 applies a normal force, which it also measures using the normal force sensor. At step 112, traction or pushing is then applied under processor control via the traction actuator. The traction sensor in sample holder 20 then observes the force that is transmitted to the other particle.

At step 114, a processor within controller 90 calculates the interparticle adhesion force and friction coefficient for the particle interaction by comparing the normal force to the traction force transmitted between the particles. The adhesion force is the minimum required force in the normal direction to separate the top particle from the bottom particle, while a friction coefficient is the ratio of friction force to the normal force applied. By varying the normal or traction force, both friction and adhesion coefficients can be calculated.

At step 116, the calculated coefficients are placed into a database, allowing statistical analysis of the coefficients. This database can include additional information about each particle, such as moisture content, if applicable. This information can be added based on the sample identifier. At step 120, the processor determines whether or not a sufficient number of samples have been taken. If not, the method returns to step 106, by prompting user to run more tests on another pair of particles or another orientation of the last particle pair. If a sufficient number of samples have been taken, at step 130, the processor identifies the most important particle interactions that contribute the most to flow restriction of the material of biomass. Results of the statistical analysis can then be output to a user. This can include a model of contribution of moisture content or humidity to the interactions.

At step 132, during real-world flow of the biomass, if material flow is unsuitable based on the statistical analysis, a material handler user can adjust ratio of particles in the biomass to adjust the fraction to result in more favorable flow characteristics. For example, if it is determined that cob to cob interaction provides the most restriction on flow, the amount of cough in biomass can be flagged as too high during material flow, prompting the user to increase the amount of other types of material in the biomass, such as leaf, to be added improve flow characteristics. Step 132 can be enhanced by using finite element analysis or a discrete element model where the flow of material in bulk solid form is modeled as a large flow of mixed particles of different types and the interparticle friction/adhesion of the individual particles is set based on a statistical model of these forces based on the experimental data.

While embodiments have been described with respect to a single exemplary design for an IPMT with a focus on certain materials, it should be appreciated that these examples are not intended to be limiting and equivalent components and different configurations can be used, including swapping the normal and traction force directions and changing which portions of the IPMT have sensors and actuators and which portions can slide or are affixed. Mechanical components can be made of any suitable material such as 3D printed plastic, molded materials, machined metal, etc. Similarly, other methods besides cyanoacrylate glues can be used to affix sample particles.

What is claimed is:

1. A measurement system for measuring flow properties of a material having a plurality of material particles comprising:
a plurality of sample holding plates, each configured to securely hold a material particle;
a first and a second sample holder, each configured to securely hold a first and second one of the plurality of sample holding plates, the first sample holder being fixed during a measurement;
a normal force actuator configured to apply a normal force between material particles held by the first and second sample holding plates;
a sliding force actuator configured to apply a sliding force between the first and second sample holding plates;
a normal force sensor configured to determine the normal force;
a sliding force sensor configured to determine an amount of sliding force transmitted from the sliding force actuator through the material particles held by the first and second sample holding plates; and
a processor configured to control the normal force and sliding force actuators, receive measurements from the normal force and sliding force sensors, and determine adhesion and friction characteristics between material particles held by the first and second sample holding plates.

2. The measurement system of claim 1, wherein each of the plurality of sample holding plates are configured to hold material particles using an adhesive.

3. The measurement system of claim 2, wherein the adhesive is a cyanoacrylate glue.

4. The measurement system of claim 1, wherein each of the plurality of sample holding plates have a mating face that is keyed such that it slides into each sample holder.

5. The measurement system of claim 1, wherein each of the plurality of sample holding plates have a mating face that is keyed such that it is configured to mount to each sample holder in a plurality of predetermined orientations to facilitate testing the same particle in multiple selectable orientations.

6. The measurement system of claim 1, wherein the processor is further configured to store the adhesion and friction characteristics for multiple combinations of the plurality of sample holding plates and to create a statistical model of interparticle forces, wherein the model divides material particles into predetermined particle types and identifies force characteristics for each particle type.

7. The measurement system of claim 1, wherein the material is corn stover and the plurality of material particles comprise pieces of cob, husk, leaf, and stalk.

8. The measurement system of claim 1, wherein the material is forest residue and the plurality of material particles comprise wood chips, bark, needles, and twigs.

9. A method for measuring flow properties of a material having a plurality of material particles, comprising steps of:
preparing a plurality of sample holding plates, each securely holding a material particle;
securing a first and second one of the plurality of sample holding plates to a first and second sample holder; and
operating a processor to:
apply a normal force between material particles held by the first and second sample holding plates using a normal force actuator,
apply a sliding force between the first and second sample holding plates using a sliding force actuator,
receive measurements from the normal force and sliding force sensors, the sliding force sensor detecting an amount of sliding force transmitted from the sliding force actuator through the material particles held by the first and second sample holding plates, and
determine adhesion and friction characteristics between material particles held by the first and second sample holding plates.

10. The method of claim 9, wherein each of the plurality of sample holding plates are configured to hold material particles using an adhesive.

11. The method of claim 10, wherein the adhesive is a cyanoacrylate glue.

12. The method of claim 9, wherein each of the plurality of sample holding plates have a mating face that is keyed such that it slides into each sample holder.

13. The method of claim 9, wherein each of the plurality of sample holding plates have a mating face that is keyed such that it is configured to mount to each sample holder in a plurality of predetermined orientations to facilitate testing the same particle in multiple selectable orientations.

14. The method of claim 9, wherein the step of operating the processor further comprises storing the adhesion and friction characteristics for multiple combinations of the plurality of sample holding plates and to create a statistical model of interparticle forces, wherein the model divides material particles into predetermined particle types and identifies force characteristics for each particle type.

15. The method of claim 9, wherein the material is corn stover and the plurality of material particles comprise pieces of cob, husk, leaf, and stalk.

16. The method of claim 9, wherein the material is forest residue and the plurality of material particles comprise wood chips, bark, needles, and twigs.

17. A measurement system for measuring flow properties of a material having a plurality of material particles comprising:
a first and second sample holder, each configured to securely hold a material particle during a measurement;
a normal force actuator configured to apply a normal force between the material particles;
a sliding force actuator configured to apply a sliding force between the material particles;
a processor configured to control the normal force and sliding force actuators, receive measurements from one or more force sensors, and determine adhesion and friction characteristics between the material particles.

18. The measurement system of claim 17, wherein the processor is further configured to store the adhesion and friction characteristics for multiple combinations of the plurality of sample holding plates and to create a statistical model of interparticle forces, wherein the model divides material particles into predetermined particle types and identifies force characteristics for each particle type.

19. The measurement system of claim 17, wherein the material is corn stover and the plurality of material particles comprise pieces of cob, husk, leaf, and stalk.

20. The measurement system of claim 17, wherein the material is forest residue and the plurality of material particles comprise wood chips, bark, needles, and twigs.
